SEQUENCE LISTING (3 sequences)

SEQ ID NO: 1 - protein, 4 aa, Artificial Sequence
  Description of Artificial Sequence Synthetic peptide
RGDC

SEQ ID NO: 2 - protein, 4 aa, Artificial Sequence
  Description of Artificial Sequence Synthetic peptide
RGDC

SEQ ID NO: 3 - protein, 4 aa, Artificial Sequence
  Description of Artificial Sequence Synthetic peptide
RGDC

(19) United States
(12) Patent Application Publication
(10) Pub. No.: US 2016/0031923 A1
(43) Pub. Date: Feb. 4, 2016

(54) GLYCOSIDE DERIVATIVES, PREPARATION THEREOF AND USE THEREOF AS PROSTHETIC GROUPS

(71) Applicants: UNIVERSITÉ DE LORRAINE, Nancy Cedex (FR); CENTRE NATIONAL DE LA RECHERCHE SCIENTIFIQUE - CNRS, Paris Cedex 16 (FR)

(72) Inventors: YVES CHAPLEUR, Nancy (FR); Sandrine LAMANDE, Vandoeuvre Les Nancy (FR); Charlotte COLLET, Gye (FR); Françoise CHRETIEN, Vandoeuvre Les Nancy (FR)

(73) Assignees: UNIVERSITE DE LORRAINE, Nancy Cedex (FR); CENTRE NATIONAL DE LA RECHERCHE SCIENTIFIQUE - CNRS, Paris Cedex 16 (FR)

(21) Appl. No.: 14/412,161

(22) PCT Filed: Jul. 2, 2013
(86) PCT No.: PCT/EP2013/063896

§ 371 Date: Jun. 24, 2015

(30) Foreign Application Priority Data
Jul. 2, 2012 (FR) 1256308

(51) Int. Cl. C07H 15/04

(52) U.S. Cl. CPC C07H 15/04

(57) ABSTRACT

The present invention relates to glycoside-derived compounds, to the processes for preparing same and to the use thereof as prosthetic groups for radiolabelling biomolecules. These compounds are co-azido-alkyl 6-deoxy-6-[¹⁸F]-fluoroglycosides of formula (I), in which: k is equal to 2 or 3; n is an integer between 1 and 5; R is independently H or a C₁-C₅ alkyl group, m being an integer between 0 and 2 if k=2 and m between 0 and 3 if k=3; and X is chosen from the group comprising O, S, CH₂ and NR′, in which R′ is independently a C₁-C₅ alkyl group or an aryl group, including all the stereoisomers thereof.

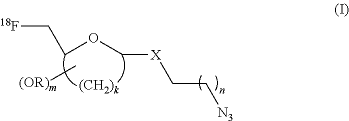

### TECHNICAL FIELD

The present invention concerns novel sugar-based compounds and more particularly glycoside derivatives, the methods of preparation thereof and their use as prosthetic groups for radiolabelling biomolecules.

### STATE OF THE ART

The monitoring and progress of molecules in the body using PET imaging (Positron Emission Tomography) requires the labelling of molecules with a positron emitting atom such as fluorine-18 (¹⁸F). The labelling of biomolecules (proteins, peptides or oligonucleotides) with ¹⁸F has been used for several years. Depending on type, biomolecules are selective for precise targets allowing highly specialised diagnosis. However, the fragility of these macromolecules does not allow the radiolabelling thereof by direct incorporation of ¹⁸F. The solution is to have recourse to a prosthetic group, a small molecule that can easily be radiolabelled and then bound to the biomolecule. At the present time several prosthetic groups are used which differ through their type and binding method. Several types of prosthetic groups are described in the literature e.g. [¹⁸F]FSB (N-succinimidyl-4-[¹⁸F]-fluorobenzoate) or [¹⁸F]FpyMe (1-[3-2(2-[¹⁸F]fluoropyridin-3-yloxy)propyl]pyrrole-2,5-dione). One major constraint in this field remains the short lifetime of the ¹⁸F atom requiring very fast reaction and purification times.

With the increasing use of these radiolabelled biomolecules there is a need to propose novel prosthetic groups that are simpler and have very fast access for the efficient radiolabelling of these biomolecules with fluorine-18.

The publication [symbol] A series of 2-O-trifluoromethylsulfonyl-_{D}-mannopyranosides as precursors for concomitant ¹⁸F-labeling and glycosylation by click chemistry [symbol] by Simone Maschauer, Olaf Prante; Carbohydrate Research 344

(2009)753-761 describes sugar-based prosthetic groups, and more particularly fluoro-sugars at 2-position and azides at anomeric position, using a [symbol] click chemistry [symbol] reaction for coupling to a biomolecule. However, it is necessary to use mannosyl precursors to obtain a glucose molecule fluorinated at 2-position. In addition, the results for labelling with fluorine 8 are only conclusive for a single type of proposed molecule (precursor 2β).

The publication [symbol] 18F-labeled glycosides for the convenient radiosynthesis of 18F-glycoconjugates by clock chemistry [symbol] by Olaf Prante and Simone Maschauer; Journal of labelled compounds and radiopharmaceuticals vol. 54, 201, page S76, describes fluoro-sugars at 2-position or 6-position and azides at anomeric position.

There is therefore a need to propose novel sugar-based molecules that can be used as simple prosthetic groups allowing very high incorporation yields of fluorine 18 independently of the geometry of the sugar, whilst maintaining the stereochemistry of the said sugar.

A further objective of the invention is to propose novel sugar-based molecules which can be used as simple prosthetic groups allowing easier radiolabelling of the biomolecules and the obtaining of improved bioavailability of the radiolabelled biomolecules so as to facilitate and improve diagnosis.

### DISCLOSURE OF THE INVENTION

For this purpose and according to the present invention there are proposed novel ω-azido-alkyl 6 deoxy-6-[¹⁸F)-fluoro-glycoside compounds of formula (I).

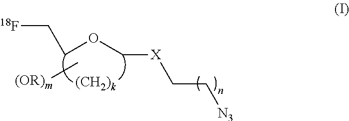

where:
k equals 2 or 3;
n is an integer between 1 and 5;
R is independently H, a C₁-C₅ alkyl group, m being an integer between 0 and 2 if k=2 and m between 0 and 3 if k=3; and
X is selected from the group comprising O, S, CH₂, NR′ where R′ is independently a C₁-C₅ alkyl group, an aryl group,
   including all the stereoisomers thereof.

### EMBODIMENTS OF THE INVENTION

More particularly, the compounds of the invention may be pentofuranose compounds and meet formula (Ia):

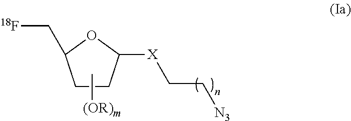

where n, R, X and m are defined above,

including all the stereoisomers thereof.

Other compounds of the invention may be hexopyranose compounds and meet formula (Ib):

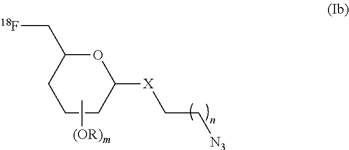

where n, R, X and m are defined above,

including all the stereoisomers thereof.

Among these compounds, preferred compounds of the invention are the compounds of formula (II):

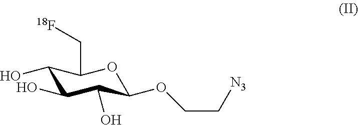

including all the stereoisomers thereof and more particularly the compounds:
containing α and β glucose (IIa):

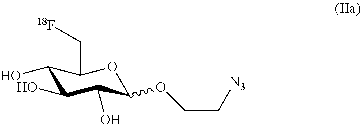

containing mannose (IIb):

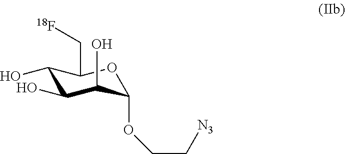

containing α and β galactose (IIc):

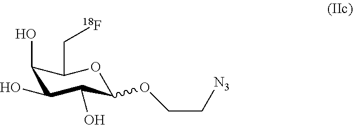

The present invention also concerns a method for synthesising a compound such as defined above, the said method comprising:
the forming, at anomeric position on a hexopyranose or pentofuranose compound, of a C₂-C₆ alkyl spacer arm terminated by an azide group;
the insertion of a leaving group at 6-position if k=3 or 5-position if k=2, and of protector groups at the other positions;
the incorporation of fluorine at 6-position if k=3 or at 5-position if k=2; and
deprotection of the other positions.

Advantageously, the insertion of a leaving group at 6-position if k=3 or at 5-position if k=2 and of protector groups at the other positions comprises the inserting of a first protector group at 6-position if k=3 or at 5-position if k=2 and of a second protector group on the other positions, the deprotection of the first protector group at 6-position if k=3 or at 5-position if k=2 by a hydroxyl group, and the insertion of the leaving group at 6-position if k=3 or at 5-position if k=2.

Preferably, the first protector group is a trityl ether and the second protector group is an acetate.

Advantageously, the leaving group is selected from the group comprising tosylate and triflate.

The tosylate leaving group can be inserted directly at 6-position if k=3 or at 5-position if k=2 without an intermediate protection step of position 6 if k=3 or of position 5 if k=2 by a trityl group.

Preferably, the leaving group is triflate.

Advantageously, the deprotection step of the other positions is performed through the action of sodium methylate with neutralisation using ascorbic acid.

According to different variants of embodiment, the hexopyranose or pentofuranose compound is selected from among hexopyranoses, pentofuranoses and the anomeric acetates thereof.

Therefore the starting reagents may be hexopyranoses and pentofuranoses to which Fischer glycosylation is applied, or glycosylations using the anomeric acetates in the presence of Lewis acids, selected for example from the group comprising BF₃-Et₂O, trimethylsilyl triflate, lanthanide salts (Yb, La, Yt, etc.) and more particularly lanthanide triflates or halides.

The present invention also concerns intermediate molecules of formula (III):

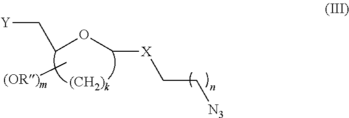

where:
Y is independently F, ¹⁸F;
R″ is selected so that OR″ forms a second protector group;
k is 2 or 3;
n is an integer between 1 and 5;
m is an integer between 0 and 2 if k=2 and between 0 and 3 if k=3;
X is selected from the group comprising O, S, CH₂, NR′ where R′ is independently a C₁-C₅ alkyl group, an aryl group;
   including all the stereoisomers thereof.

Among these compounds, preferred intermediate molecules of the invention are the intermediate molecules of formula (IV):

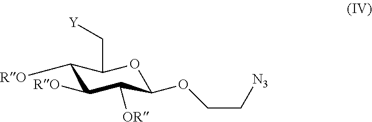

where:
R″ is an acetyl group;
Y is independently F, ¹⁸F;
   including all the stereoisomers thereof.

The present invention also concerns the intermediate molecules of formula (V):

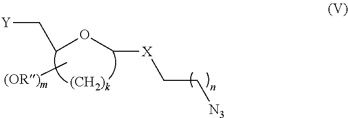

where:
Y is independently a tosylate leaving group, a triflate leaving group;
R″ is an acetyl group;
k is 2 or 3;
n is an integer between 1 and 5;
m is an integer between 0 and 2 if k=2 and between 0 and 3 if k=3;
X is selected from the group comprising O, S, CH₂, NR′ where R′ is independently a C₁-C₅ alkyl group, an aryl group;
   including all the stereoisomers thereof.

Among these compounds, preferred intermediate molecules of the invention are the intermediate molecules of formula (VI):

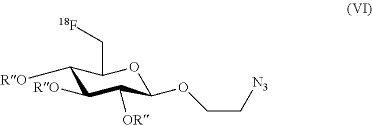

where:
R″ is an acetyl group;
Y is independently a tosylate leaving group, a triflate leaving group;
   including all the stereoisomers thereof.

Preferably, the second protector group is an acetate.

The present invention concerns the compounds of formula I, Ia, Ib, II, IIa, IIb, IIc for use as prosthetic group intended to be coupled to a biomolecule via cycloaddition of its azide group with a terminal alkyne group provided on said biomolecule, as per a coupling reaction via click chemistry. Such reactions, of Huisgen type, are known to persons skilled in the art and will not be described in detail herein.

The present invention also concerns the use of a compound of formula I, Ia, Ib, II, IIa, IIb, IIc to radiolabel a biomolecule on which a terminal alkyne group is provided.

Such biomolecules are proteins, peptides or oligonucleotides for example which can be modified to insert a terminal alkyne function. More specifically, said biomolecules may be peptides comprising the Arginine-Glycine-Aspartic Acid or bombesin sequence to detect cancers, peptides involved in inflammation such as those described in application WO 2005/071408, or antibody chains.

Evidently the present invention encompasses all the stereoisomers and optical isomers of all the illustrated formulas, whether pure or in a mixture.

The compounds of the invention allow:
the use of methods, even automated equipment, already used for the preparation of (¹⁸F]fluorodeoxyglucose (FDG) a compound routinely used in all PET imaging units;
advantage to be taken of the improvement in the pharmacological parameters of the radiotracer through the presence of the sugar (increase in hydrophilicity, reduction in binding to plasma proteins, reduction in liver uptake and specific uptakes, improved tumour uptake and kidney excretion, etc.).

The method of the invention has the advantage of being able to be applied to all hexopyranoses and pentofuranoses and in particular to a common hexopyranose such as glucose and has the ability to maintain the stereochemistry of the said sugar.

In addition, the method of the invention allows very high incorporation yields of fluorine 18 to be obtained independently of the geometry of the sugar, which is of prime importance for radiosynthesis. More particularly, the incorporation yields of fluorine 18 are high and similar whether on mannose, glucose at position α or β or galactose at position α or β.

The use of sugar-based prosthetic groups has the advantage of increasing the bioavailability of the radiolabelled biomolecule and thereby allows better distribution and the obtaining of improved PET images.

In addition, the coupling method using click chemistry allows coupling reactions to be conducted within a very short time and with very good yields, of essential importance in radiochemistry. Therefore, all the synthesis and radiosynthesis steps of the method of the invention and the coupling steps on the biomolecule allow high yields to be obtained comparable with those obtained using standard prosthetic groups.

The following examples illustrate the present invention without however limiting the scope thereof.

1/ Synthesis of the Prosthetic Groups

The scheme below gives the general synthesis of 2′-azidoethyl-6-fluoro-glyopyranoside derivatives via glycosylation using the anomeric acetate:

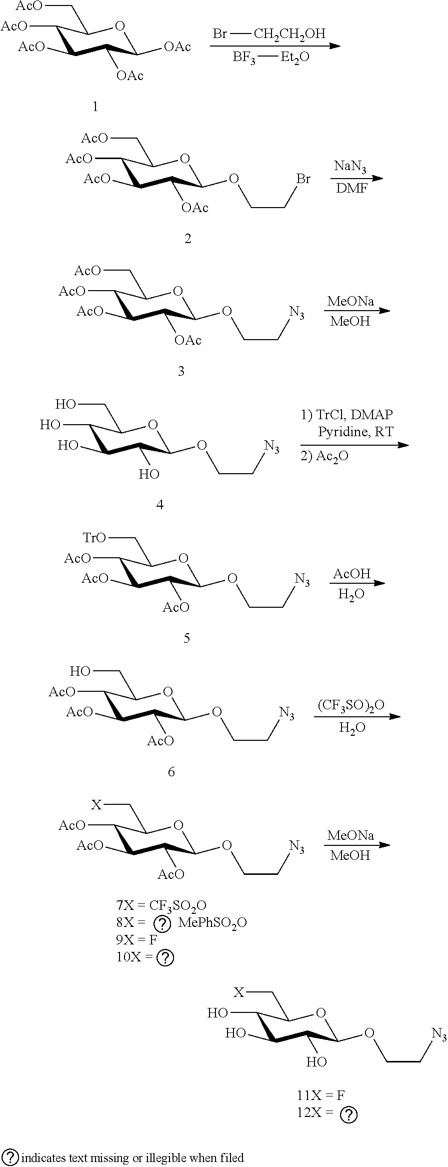

According to another variant, compound 2 can be obtained from the corresponding sugar via Fischer glycosylation according to an example below:

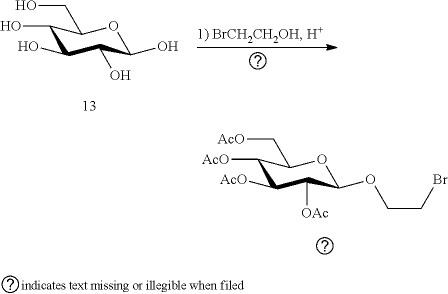

In the examples of synthesis given below, for each step a general operating mode is indicated for a compound referenced X, this operating mode being valid for all the corresponding stereoisomers obtained and referenced Xa, Xb, Xc, the stereoisomers then being described separately.

General Operating Mode for the Synthesis of Compounds 2

1) Glycosylation via the Anomeric Acetates

To a solution of compound 1 (7.8 g, 20 mmol) in 210 mL of dry dichloromethane in a 500 mL round-bottomed flask supplied with a flow of nitrogen and cooled to 0° C. are added 2-bromo-ethanol (2 equivalents, 5 g) and then dropwise boron trifluoride etherate (4 equivalents, 11.4 mL) under vigorous agitation. The solution is slowly heated to ambient temperature in 3 h. Analysis by thin layer chromatography (TLC) eluting with a toluene-ethyl acetate mixture 2:1 shows that the reaction is complete. Triethylamine is added dropwise to the medium until decolouring of the mixture. The mixture is diluted with dichloromethane (300 mL). The organic phase is washed in water (2×50 mL) and dried over magnesium sulphate. The residue obtained after evaporation of the solvent is purified on a silica column (eluting with cyclohexane-ethyl acetate 7:3) to give compound 2.

The following compounds are thus obtained:
2′-bromoethyl-2,3,4,6-tetra-O-acetyl-β-D-glucopyranoside (2a) (7.75 g, 85%);
2′-bromoethyl-2,3,4,6-tetra-O-acetyl-α-D-mannopyranoside (2b) (4.92 g 54%);
2′-bromoethyl-2,3,4,6-tetra-O-acetyl-β-D-galactopyranoside (2c-β) (4.55 g 50%).

2) Fischer Glycosylation

To a solution of glucose or galactose compound (5 g, 27.7 mmol) in 50 mL of 2-bromo-ethanol in a 100 mL round-bottomed flask supplied with a flow of nitrogen are added 5 g of Amberlite IR-120 under strong agitation. The solution is heated to 70° C. for 4 h. The solution is then cooled to ambient temperature, filtered and rinsed with ethanol. The reaction mixture is then evaporated under reduced pressure. The residue obtained is purified by flash chromatography (eluting from 100% ethyl acetate to 80:20 ethyl acetate-methanol to remove the sugar that has not reacted. The α/β mixture is obtained with 60% yield in the gluco series or 52% in the galacto series.

To a solution of the preceding compound (4.1 g, 14.3 mmol) in 200 mL of pyridine under a flow of nitrogen are added 0.1 equivalent (eq) (430 mg, 3.5 mmol) of N,N-dimethylamino-4 pyridine (DMAP) and acetic anhydride (50 mL). The solution is left under agitation for 1 h at ambient temperature. The reaction mixture is then concentrated under reduced pressure and co-evaporated with tolene. The residue is re-dissolved in dichloromethane (300 mL) washed in water (50 mL), dried over magnesium sulphate and evaporated. The crude is purified on a silica column (cyclohexane/ethyl acetate: 60:40) to give compound 2.

For example the following compounds are obtained: 2′-bromoethyl-2,3,4,6-tetra-O-acetyl-α-D-galactopyranoside (2c-α) (70%) and compound 2′-bromoethyl-2,3,4,6-tetra-O-acetyl-β-D-galactopyranoside (2c-β) (30%). The compound 2′-bromoethyl-2,3,4,6-tetra-O-acetyl-α-D-glucopyranoside (2a-α) is obtained with a yield of 63%.

General Operating Mode for the Synthesis of Compounds 3

To a solution of compound 2 (7 g, 15.4 mmol) in 200 mL of dimethylformamide in a 500 mL round bottomed flask supplied with a flow of nitrogen the addition is made of sodium azidide (3 equivalents, 3 g) under strong agitation. The solution is heated to 90° C. for 1 h. Analysis by thin layer chromatography (TLC) eluting with a 3:1 mixture of toluene and ethyl acetate shows that the reaction is complete. The mixture is concentrated in vacuo and the residue re-dissolved in dichloromethane (300 mL). The organic phase is washed in water (50 mL) then washed with a 3 M HCl solution (5 ml) and then water (2×50 mL), and dried over magnesium sulphate. The residue obtained after evaporation of the solvent is purified on a silica column (eluting with cyclohexane/ethyl acetate 7:3) to give compound 3.

The following compounds are thus obtained:
2′-azidoethyl-2,3,4,6-tetra-O-acetyl-β-D-glucopyranoside (3a-β) (4.8 g, 69%);
2′-azidoethyl-2,3,4,6-tetra-O-acetyl-α-D-mannopyranoside (3b) (4.5 g 67%);
2′-azidoethyl-2,3,4,6-tetra-O-acetyl-β-D-galactopyranoside (3c-β) (3.5 g 51%);
2′-azidoethyl-2,3,4,6-tetra-O-acetyl-α-D-galactopyranoside (3c-α) (5.4 g 78%);
   The compound 2′azidoethyl-2,3,4,6-tetra-O-acetyl-α-D-glucopyranoside (3a-α) is obtained with a yield of 68%.

General Operating Mode for the Synthesis of Compounds 4

To a solution of compound 3 (7.76 g, 18.6 mmol) in 110 mL of anhydrous methanol are added 2.5 mL of 1 M sodium methylate solution. The medium is left under agitation at ambient temperature for 2 hours then neutralised with the addition of resin (Amberlite IR-120), filtered and evaporated. The products are used directly at the following step.
2′-azidoethyl-β-D-glucopyranoside (4a-β) (yield: 98%, clear oil);
2′azidoethyl-α-D-mannopyranoside (4b) (yield: 95%, pale yellow solution);
2′-azidoethyl-β-D-galactopyranoside (4c-β) (yield: 99% clear oil);
2′-azidoethyl-α-D-galactopyranoside (4c-α) (yield 99%, clear oil);
   The compound 2′-azidoethyl-α-D-glucopyranoside (4a-α) is obtained with a yield of 96%.

General Operating Mode for the Synthesis of Compounds 5

To a solution of compound 4 (8.6 g, 35 mmol) in 200 mL of pyridine supplied with a flow of nitrogen are added 1.8 equivalents (17.61 g, 63 mmol) of trityl chloride and 0.1 eq (430 mg, 3.5 mmol) of DMAP. The mixture is left under agitation at ambient temperature and the reaction monitored by TLC. When the reaction is complete, acetic anhydride is added (45 mL) and the solution left under agitation for 3 h. The reaction mixture is then concentrated in vacuo and co-evaporated with toluene. The residue is re-dissolved in dichloromethane (300 mL) and washed in water (50 mL). The organic phase is washed with 1 M HCl solution (5 mL), water (2×50 mL), 3M sodium hydroxide (5 mL), water (2×50 mL), then dried over magnesium sulphate and evaporated. The crude is purified on a silica column (hexane/ethyl acetate: 4:6).

The following compounds are obtained:

### 2′-azidoethyl-6-O-trityl-2,3,4-tri-O-acetyl-β-D-glucopyranoside 5aβ

Yield: 64%; R_{f} 0.45 (Hexane/AcOEt 60:40); white solid; mp=68° C.; [α]_{D}=+9.8 (1, CH₂Cl₂); IR: film (v, cm⁻¹): 2881, 2104, 1757, 1219; ^{1H}NMR (CDCl₃, 400 MHz) δ (ppm): 1.75 (s, 3H, OAc), 2.00 (s, 3H, OAc), 2.07 (s, 3H, OAc), 3.12 (dd, 1H, J_{6,5}=4.8 Hz, J_{6,6′}=10.5 Hz, H6), 3.8 (dd, 1H, J_{6′,5}=2.3 Hz, H6′), 3.30-3.35 (m, 1H, H8), 3.50-3.62 (m, 2H, H5, H8′), 3.78 (ddd, 1H, J_{7,8′}=3.0 Hz, J_{7,8}=7.9 Hz, J_{7,7′}=10.9 Hz, H7), 4.05-4.15 (m, 1H, H7′), 4.61 (d, 1H, J_{1,2}=7.5 Hz, H1, 5.05-5.22 (m, 3H, H2, H3, H4), 7.20-7.34 (m, 10H, H—Ar), 7.42-7.48 (m, 5H, H—Ar); ^{13C}NMR, (CDCl₃, 100 MHz) δ (ppm): 20.4 (OAc), 20.6 (OAc), 20.7 (OAc), 50.6 (C-8), 61.9 (C-6), 68.2 (C-7), 68.7 (C-4), 71.3 (C-2), 73.1 (C-5), 73.5 (C-3), 86.6 (Cq Tr), 100.8 (C-1), 127.1 (3C—Ar), 127.8 (6C—Ar), 128.7 (6C—Ar), 143.5 (3Cq-Ar), 168.9 (C═O), 169.5 (C═O), 170.4 (C═O); MS (ESI), 640 (M+Na)⁺; Analysis: calculated for C₃₃H₃₅N₃O₉: C: 64.17; H: 5.71; N: 6.80. Found: C: 64.29; H: 5.80; N: 6.61.

### 2′-azidoethyl-6-O-trityl-2,3,4-tri-O-acetyl-α-D-glucopyranoside 5aα

Yield: 84%; R_{f} 0.45 (Cyclohexane/AcOEt 60:40) ; white solid; mp=117° C.; [α]_{D}=+108.8 (0.5, CHCl₃); IR (film): v (cm⁻I): 2934, 2107, 1754, 224, 1043; ^{IH}NMR (CDCl₃, 400MHz): δ (ppm): 1.76 (s, 3H, OAc), 2.02 (s, 3H, OAc), 2.11 (s, 3H, OAc), 3.14 (dd, 1H, J_{6,6′}=10.5 Hz, J_{6,5}=5.0 Hz, H-6), 3.23 (dd, 1H, J_{6′,5}=2.0 Hz, H-6′), 3.43 (ddd, 1H, J_{8,8′}=13.5 Hz, J_{8,7′}=6.0 Hz, J_{8,7}=3.5 Hz, H-8), 3.53 (ddd, 1H, J_{8′,7}=7.0 Hz, J_{8′,7′}=3.5 Hz, H-8′), 3.70 (ddd, 1H, J_{7,7′}=10.5 Hz, H-7), 3.95-4.02 (m, 2H, H-5 and H-7′), 4.95 (dd, 1H, J_{2,3}=10.0 Hz, J_{2,1}=4.0 Hz, H-2), 5.12 (app t, 1H, J_{4,3}=J_{4,5}=10.0 Hz, H-4), 5.22 (d, 1H, H-1), 5.48 (ta, 1H, H-3), 7.25 (tl, 3H, J=7.0 Hz, H—Ar), 7.31 (tl, 6H, J=7.0 Hz, H—Ar), 7.45 (dl, 6H, J=7.0, Hz, H—Ar); ^{13C}NMR (CDCl₃, 100.6 MHz: δ (ppm): 20.5 (OAc), 20.7 (OAc), 20.7 (OAc), 50.4 (C8), 62.0 (C-6), 67.0 (C-7), 69.0 (C-4), 69.1 (C-5), 70.4 (C-3), 70.9 (C-2), 86.6 (Cq-Tr), 95.8 (C-1), 127.0 (3C—Ar), 127.8 (6C—Ar), 128.7 (6C—Ar), 143.6 (3C—Ar), 169.3 (C═O), 170.2 (C═O), 170.4 (C═O); MS (HR-ESI) calculated for C₃₃H₃₅N₃O₉Na [M+Na]⁺ 640 266. Found: 640 2277.

### 2′azidoethyl-6-O-trityl-2,3,4-tri-O-acetyl-α-D-mannopyranoside 5b

Yield: 67%; R_{f} 0.4 (Cyclohex/AcOEt 60:40); white solid, mp=58° C.; [α]_{D}=+49.3 (1, CHCl₃); IR: film (v, cm⁻¹): 3059, 2932, 2105, 1748, 1371; ^{IH}NMR (CDCl₃, 400 MHz) δ (ppm): 1.76 (s, 3H, OAc), 1.99 (s, 3H, OAc), 2.16 (s, 3H, OAc), 3.19 (dd, 1H, J_{6,6′}=10.5 Hz, J_{5,6} =5.3 Hz, H6), 3.23 (dd, 1H, J_{5,6′}=2.4 Hz, H6′), 3.41-3.55 (m, 2H, H8), 3.72 (ddd, 1H, J_{7,7′}=10.3 Hz, J_{7,8}=6.3 Hz, J_{7,8′}=3.9 Hz, H7), 3.95-4.00 (m, 2H, H5, H7′), 4.94 (d, 1H, J_{1,2}=1.5 Hz, H1), 5.27-5.37 (m, 3H, H2, H3, H4), 7.21-7.35 (m, 9H, H—Ar), 7.46 (dd, 6H, J=8.5 Hz, J=1.1 HZ, H—Ar); ^{13C}NMR, (CDCl₃, 100 MHz) δ (ppm): 21.0 (OAc), 21.1 (OAc), 21.3 (OAc), 50.8 (C-8), 62.8 (C-6), 66.8 (C-4), 67.1 (C-7), 69.6 (C-3 or C-2), 70.1 (C-3 or C-2), 71.0 (C-5), 87.0 (Cq Tr), 97.9 (C-1), 127.4 (3C—Ar), 128.2 (6C—Ar), 129.1 (6C—Ar), 144.1 (Cq-Ar), 169.8 (C═O), 170.4 (C═O), 170.5 (C═O); MS (HR-ESI) calculated for C₃₃H₃₅N₃O₉Na [M+Na]⁺ 640 2271. Found: 640 2336. Analysis: calculated for C₃₃H₃₅N₃O₉: C: 64.17; H: 5.71; N: 6.80. Found: C: 64.30; H: 5.74; N: 6.69.

### 2′-azidoethyl-6-O-trityl-2,3,4-tri-O-acetyl-α-D-galactopyranoside 5cα

Yield: 58%; R_{f} 0.4 (Cyclohexane/AcOEt 60:40); white solid; _{IH}NMR (CDCl₃, 400 MHz) δ (ppm): 1.99 (s, 3H, OAc), 2.05 (s, 3H, OAc), 2.08 (s, 3H, OAc), 2.15 (s, 3H, OAc), 3.39 (ddd, 1H, J_{8,8′}=13.5 Hz, J_{7′,8}=6.0 Hz, J_{7,8}=3.5 Hz, H8), 3.49 (ddd, 1H, J_{7,8′}=7.0 Hz, J_{7′,8′}=3.5 Hz, H8′), 3.63 (ddd, 1H, J_{7,7′}=11.0 Hz, H7), 3.87 (ddd, 1H, H7′), 4.10 (app d, 2H, J_{5,6}=J_{6,6′}=6.5 Hz, H6), 4.10 (app dt, 1H, J_{4,5}=1.0 Hz, H5), 5.14 (dd, 1H, J_{2,3}=10.5 Hz, J_{1,2}=4.0 Hz, H2), 5.17 (d, 1H, H1), 5.37 (dd, 1H, J_{3,4}=3.5 Hz, H3), 5.48 (dd, 1H, H4); ^{13C}NMR (CDCl₃, 62.9 MHz) δ (ppm): 20.8 (2OAc), 20.8 (OAc), 20.9 (OAc), 50.6 (C-8), 61.9 (C-6), 66.7 (C-5), 67.5 (C-7), 67.6 (C-3), 68.0 (C-2), 68.2 (C-2), 96.7 (C-1), 170.1 (C═O), 170.3 (C═O═, 170.5 (C═O), 170.7 (C═O).

### 2′azidoethyl-6-O-trityl-2,3,4-tri-O-acetyl-β-D-galactopyranoside 5cβ

Yield: 64%; R_{f} 0.45 (Cyclohexane/AcOEt 60:40); white solid; [α]_{D}=+56.3 (1, CH₂Cl₂); IR: film (v, cm⁻¹): 2933, 2099, 1747, 1222, ^{IH}NMR (CDCl₃, 400 MHz) δ (ppm): 1.90 (s, 3H, OAc), 1.99 (s, 3H, OAc), 2.06 (s, 3H, OAc), 3.11 (app t, 1H, J_{6,6′}=8.5 Hz, J_{5,6′}=8.0 Hz, H6′), 3.26 (ddd, 1H, J_{8,8′}=13.5 Hz, J_{7′,8}=4.5 Hz, J_{7,8}=3.5 Hz, H8), 3.39 (dd, 1H, J_{5,6}=6.0 Hz, H6), 3.45 (ddd, 1H, J_{7,8′}=8.5 Hz, J_{7′,8′}=3.5 Hz, H8′), 3.64 (ddd, 1H, J_{7,7′}=10.5 Hz, H7), 3.82 (ddd, 1H, J_{4,5}=0.5 Hz, H5), 4.01 (ddd, 1H, H7′), 4.53 (d, 1H, J_{1,2}=8.0 Hz, H1), 5.06 (dd, 1H, J_{2,3}=10.5 Hz, J_{3,4}=3.5 Hz, H3), 5.18 (dd, 1H, H2), 5.57 (dd, 1H, H4), 7.26 (bt, 3H, J=7.5 Hz, H—Ar), 7.30 (bt, 6H, J=7.5 Hz, H—Ar), 7.37 (bd, 6H, J=7.5 Hz, H—Ar); ^{13C}NMR (CDCl₃, 100 MHz) δ (ppm): 20.7 (OAc), 20.8 (OAc), 20.9 (OAc), 50.7 (C-8), 60.9 (C-6), 67.4 (C-4), 68.4 (C-7), 70.0 (C-2), 71.3 (C-3), 72.4 (C-5), 87.0 (Cq Tr), 101.2 (C-1), 127.3 (3C—Ar), 128.0 (6C—Ar), 128.7 (6C—Ar), 143.4 (3Cq-Ar), 169.7 (C═O), 170.0 (c=O), 170.3 (C═O).

General Operating Mode for the Synthesis of Compounds 6

A solution of 25 mmol (15.38 g) of compound 5 in 250 mL of AcOH/H2O mixture (3:1) is heated to 80° C. for 1 h 30. At the end of the reaction monitored by TLC the reaction mixture is concentrated under reduced pressure then co-evaporated 4 times with 10 mL of toluene. The crude is purified on a silica column (hexane/ethyl acetate 50:50 then 30:70).

The following compounds are obtained:

### 2′-azidoethyl-2,3,4-tri-O-acetyl-β-D-glucopyranoside 6aβ

Yield: 54%; R_{f} 0.2 (Hex/AcOEt 60:40); white solid; mp=112° C.; [α]_{D}=−42.0 (1, CH₂Cl₂); IR: film (v, cm⁻¹): 3457, 2924, 2105, 1755, 1218; ^{IH}NMR (CDCl₃, 400 MHz) δ (ppm): 2.01 (s, 3H, OAc), 2.05 (s, 6H, 2OAc), 3.29 (ddd, 1H, J_{8,7}=3.4 Hz, J_{8,7′}=5.1 Hz, J_{8,8′}=13.4 Hz, H8), 3.40-3.78 (m, 5H, H5, 2H6, H7, H8′), 4.04 (ddd, 1H, J_{7′,8′}=3.4 Hz, J_{7′,7}=10.6 Hz, H7′), 4.63 (d, 1H, J_{1,2}=7.9 Hz, H1), 4.97-5.09 (m, 2H, H4, H2), 5.27 (app t, 1H, J_{2,3}=J_{3,4}=9.5 Hz, H3); ^{13C}NMR (CDCl₃), 100 MHz) δ (ppm): 20.5 (OAc), 20.6 (OAc), 20.6 (OAc), 50.5 (C-8), 61.2 (C-6), 68.4 (C-7), 68.6 (C-4), 71-2 (C-2), 72.7 (C-5), 74.2 (C-3), 100.6 (C-1), 169.4 (C═O), 170.1 (C═O), 170.2 (C═O); MS ESI, 398 (M+Na)⁺. Analysis: Calculated for C₁₄H₂₁N₃O₉: C: 44.80; H: 5.63; N: 11.19. Found: C: 45.19; H: 5.75; N: 10.88.

### 2′-azidoethyl-2,3,4-tri-O-acetyl-α-D-glucopyranoside 6aα

Yield: 82%; R_{f} 0.45 (Cyclohexane/AcOEt 60:40); white solid; mp=79° C.; [α]_{D}=+134.3 (0.5, CHCl₃); IR (film): v (cm⁻¹): 3483, 2928, 2109, 1751, 1370, 1226, 1039; ^{IH}NMR (CDCl₃, 400 MHz): δ (ppm): 2.02 (s, 3H, OAc), 2.06 (s, 3H, OAc), 2.07 (s, 3H, OAc), 3.39 (ddd, 1H, J_{8,8′}=13.5 Hz, J_{8,7′}=6.0 Hz, J_{8,7}=3.5 Hz, H-8), 3.48 (ddd, 1H, J_{8′,7}=7.0 Hz, J_{8′,7}=3.5 Hz, H-8′), 3.56-3.61 (m, 1H, H-6), 3.62 (ddd, 1H, J_{7,7′}=10.5 HZ, H-7), 3.71 (ddl, 1H, J_{6,6′}=12.5 Hz, J_{6,5}=7.5 Hz, H-6′), 3.81-3.86 (m, 1H, H-5), 3.87 (ddd, 1H, H-7′), 4.85 (dd, 1H, J_{2,3}=10.0 Hz, J_{2,1}=3.5 Hz, H-2), 5.02 (app t, 1H, J_{4,3}=J_{4,5}=10.0 Hz, H-4), 5.14 (d, 1H, H-1), 5.56 (app t, 1H, H-3); ^{13C}NMR (CDCl₃, 100.6 MHz): δ (ppm): 20.6 (2OAc), 20.7 (OAc), 50.4 (C-8), 61.0 (C-6), 67.3 (C-7), 68.8 (C-4), 69.6 (C-3), 69.7 (C-5), 70.8 (C-2), 96.0 (C-1), 170.0 (C═O), 170.4 (C═O), 170.6 (C═O); MS (HR-ESI) calculated for C₁₄H₂₁N₃O₉Na[M+Na]⁺ 398.1170. Found: 398.1172.

### 2′-azidoethyl-2,3,4-tri-O-acetyl-α-D-mannopyranoside 6b

Yield: 75%; R_{f} 0.15 (Hex/AcOEt 50:50); oil; [α]_{D}=+44.8 (1, CHCl₃); IR; film (v, cm⁻¹): 3469, 2107, 1752, 1639, 1371; ^{1H}NMR(CDCl₃, 400 MHz) δ (ppm): 1.99 (s, 3H, OAc), 2.08 (s, 3H, OAc), 2.14 (s, 3H, OAc), 3.35-3.55 (m, 2H, H8), 3.60-3.70 (m, 3H, 2H6, 1H7), 3.80-3.95 (m, 2H, H5, H7′), 4.89 (d, 1H, J_{1,2}=1.6 Hz, H1), 5.25 (app t, 1H, J_{4,5}=10.2 Hz, H4), 5.28 (dd, 1H, J_{2,3}=3.4 Hz, H2), 5.40 (dd, 1H, H3); ^{13C}NMR (CDCl₃, 100 MHz) δ (ppm): 21.1 (OAc), 21.1 (OAc), 21.2 (OAc), 50.7 (C-8), 61.6 (C-6), 66.7 (C-4), 67.3 (C-7), 69.0 (C-3), 69.8 (C-2), 71.4 (C-5), 98.1 (C-1), 170.2 (C═O), 170.4 (C═O), 171.3 (C═O); MS ESI, 398 (M+Na)⁺; Analysis: Calculated for C₁₄H₂₁N₃O₉: C: 44.80; H: 5.63; N: 11.19. Found: C: 44.02; H: 5.64; N: 10.71.

### 2′-azidoethyl-2,3,4-tri-O-acetyl-α-D-galactopyranoside 6cα

Yield: 71%; R_{f} 0.18 (Cyclohexane/AcOEt 50:50); colourless oil; [α]_{D}=−6.6 (1, CH₂Cl₂); IR: film (v, cm⁻¹): 3483, 2937, 2109, 1747, 1230; ^{1H}NMR (Acetone-D6, 400 MHz) δ (ppm): 1.92 (s, 3H, OAc), 2.03 (s, 3H, OAc), 2.11 (s, 3H, OAc), 3.48 (ddd, 1H, J_{8,8′}=13.5 Hz, J_{7′,8}=6.0 Hz, J_{7,8}=3.5 Hz, H8), 3.52-3.65 (m, 3H, 2H6 and H8′), 3.69 (ddd, 1H, J_{7,7′}=11.0 Hz, J_{7,8′}=7.0 Hz, H7), 3.92 (dd, 1H, J_{6,OH}=5.0 Hz and J_{6′,OH}=7.0 Hz, OH), 3.96 (ddd, 1H, J_{7′,8′}40 =3.5 Hz, H7′), 4.15 (app dt, 1H, J_{5,8}=7.0 Hz, J_{4,5}=1.0 Hz, H5), 5.08-dd, 1H, J_{2,3}=11.0 Hz, J_{1,2}=3.5 Hz, H2), 5.16 (d, 1H, H1), 5.32 (dd, 1H, J_{3,4}=3.5 Hz, H3), 5.51 (dd, 1H, H4); ^{13C}NMR (Acetone-D6, 100 MHz) δ (ppm): 20.6 (OAc), 20.7 (OAc), 20.7 (OAc), 51.2 (C-8), 60.9 (C-6), 68.0 (C-7), 68.7 (C-3), 68.9 (C-2), 69.1 (C-4), 70.3 (C-5), 97.2 (C-1), 170.3 (C═O), 170.8 (C═O), 170.8 (C═O).

### 2′-azidoethyl-2,3,4-tri-O-acetyl-β-D-galactopyranoside 6cβ

Yield: 72%; R_{f} 0.20 (Cyclohexane/AcOEt 50:50); white solid; [α]_{D}=+134.41 (1, CH₂Cl₂); IR; film (v, cm⁻¹): 3440, 2924, 2099, 1747, 122; ^{1H}NMR (Acetone-D6, 400 MHz) δ (ppm): 1.91 (s, 3H, OAc), 2.01 (s, 3H, OAc), 2.11 (s, 3H, OAc), 3.39 (ddd, 1H, J_{8,8′}=13.5 Hz, J_{7′,8}=5.5 Hz, J_{7,8}=3.5 Hz, H8), 3.50 (ddd, 1H, J_{7,8′}=8.0 Hz, J_{7′,8′}=3.5 Hz, H8′), 3.56 (app dt, 1H, J_{6,6′}=11.0 Hz, J_{5,6}=7.0 Hz, J_{6,OH}=7.0 Hz, H6), 3.67 (ddd, 1H, J_{5,6′}=7.0 Hz, J_{6′,OH}=5.0 Hz, H6′), 3.75 (ddd, 1H, J_{7,7′}=11.0 Hz, H7), 3.95 (dd, 1H, OH), 3.96 (app dt, 1H, J_{4,5}=1.0 Hz, H5), 4.03 (ddd, 1H, H7′), 4.76 (d, 1H, J_{1,2}=8.0 Hz, H1), 5.08 (dd, 1H, J_{2,3}=10.5 Hz, J_{3,4}=3.5 Hz, H3), 5.15 (dd, 1H, H2), 5.44 (dd, 1H, H4); ^{13C}NMR (Acetone-D6, 100 MHz) δ (ppm): 20.6 (OAc), 20.6 (OAc), 20.8 (OAc), 51.4 (C-8), 60.8 (C-6), 68.5 (C-4), 69.0 (C-7), 69.8 (C-2), 72.2 (C-3), 74.6 (C-5), 101.8 (C-1), 169.8 (C═O), 170.3 (C═O), 170.9 (C═O).

General Operating Mode for the Synthesis of Compounds 7 Using a Triflate as Leaving Group.

To a solution of 0.47 mmol (150 mg) of compound 6 in 2 mL of pyridine cooled to −25° C. is added dropwise 1 eq (83 μL, 0.47 mmol) of trifyl anhydride. The solution is left under agitation 10 min at −25° C. then brought to ambient temperature for 10 min. The reaction mixture is hydrolysed then extracted with CH₂Cl₂ (100 mL), washed in a saturated aqueous NaHCO₃ solution (2×5 mL) then with water (10 mL). The organic phase is dried over MgSO₄ then evaporated under reduced pressure. The crude is purified on a silica column (hexane/ethyl acetate 60:40).

The following compounds are obtained:

### 2′-azidoethyl-6-O-triflate-2,3,4-tri-O-acetyl-β-D-glucopyranoside 7aβ

Yield: 74%; R_{f} 0.45 (Hex/AcOEt 60:40); white solid, mp=101° C.; [α]_{D}=−42.7 (1, CHCl₃); IR: film (v, cm⁻¹): 2945, 2107, 1759, 1417, 1376, 1215; ^{1H}NMR (CDCl₃, 400 MHz) δ (ppm): 2.02 (s, 3H, OAc), 2.06 (s, 3H, OAc), 2.07 (s, 3H, OAc), 3.30 (ddd, 1H, J_{8,8′}=13.4 Hz, J_{8,7′}=4.7 Hz, J_{8,7}=3.2 Hz, H8), 3.51 (ddd, 1H, J_{8′,7}=8.5 Hz, J_{8′,7′}=3.2 Hz, H8′), 3.70 (ddd, 1H, J_{7,7′}=10.7 Hz, H7), 3.87 (ddd, 1H, J_{5,4}=9.4 Hz, J_{5,6′}=6.4 Hz, J_{5,6}=2.7 Hz, H5), 4.05 (ddd, 1H, H7′), 4.50 (dd, 1H, J_{6,6″}=11.4 Hz, H6), 4.57 (dd, 1H, J_{6′,6}=11.4 Hz, J_{6′,5}=6.4 Hz, H6′), 4.65 (d, 1H, J₁,=8.0 Hz, H1), 4.98 (app t, 1H, J_{3,4}=9.4 Hz, H4), 5.03 (dd, 1H, J_{2,3}=9.4 Hz, H2), 5.26 (app t, 1H, H3); ^{13C}NMR (CDCl₃, 100 MHz) δ (ppm): 20.9 (OAc), 20.9 (OAc), 21.0 (OAc), 50.9 (C-8), 68.8 (C-4 or C-7), 69.1 (C-4 or C-7), 71.2 (C-2), 71.9 (C-5), 72.6 (C-3), 73.9 (C-6), 100.9 (C-1), 118.9 (q, J_{C,F}=320.0 Hz, C-Tf), 169.7 (C═O), 170.0 (C═O), 70.5 (C═O); ^{19F}NMR (CDCl₃, 235 MHz) δ (ppm): −74.7; MS (HR-ESI) calculated for C₁₅H₂₀N₃O₁₁F₃SNa [M+Na]⁺ 530.0668. Found: 530.0660.

### 2′-azidoethyl-6-O-triflate-2,3,4-tri-O-acetyl-α-D-glucopyranoside 7aα

Yield: 34%; R_{f} 0.39 (Cyclohexane/AcOEt 60:40); colourless oil; [α]_{D}=+70.3 (0.6, CHCl₃); IR (film): v (cm⁻¹): 2937, 2109, 1755, 1417, 1219, 1145, 1038; ^{IH}NMR (CDCl₃, 400 MHz): δ (ppm): 2.02 (s, 3H, OAc), 2.07 (s, 3H, OAc), 2.08 (s, 3H, OAc), 3.43 (ddd, 1H, J_{8,8′}=13.5 Hz, J_{8,7′}=6.0 Hz, J_{8,7}=3.5 Hz, H-8), 3.50 (ddd, 1H, J_{8′,7}=7.0 Hz, J_{8′,7′}=3.0 Hz, H-8′), 3.65 (ddd, 1H, J_{7,7′}=11.0 Hz, H-7), 3.88 (ddd, 1H, H-7′), 4.19 (ddd, 1H, J_{5,4}=10.5 Hz, J_{5,6′}=6.0 Hz, J_{5,6}=2.5 Hz, H-5), 4.49 (dd, 1H, J_{6,6′}=11.0 Hz, H-6), 4.54 (dd, 1H, H-6′), 4.87 (dd, 1H, J_{2,3}=10.5 Hz, J_{2,1}=3.5 Hz, H-2), 4.98 (dd, 1H, J_{4,5}=10.5 Hz, J_{4,3}=9.5 Hz, H-4), 5.16 (d, 1H, H-1), 5.53 (dd, 1H, H-3); ^{13C}NMR (CDCl₃, 100.6 MHz): δ (ppm): 20.5 (OAc), 20.6 (OAc), 20.6 (OAc), 50.46 (C-8), 67.3 (C-5), 67.6 (C-7), 68.4 (C-4), 69.5 (C-3), 70.3 (C-2), 73.6 (C-6), 95.9 (C-1), 118.5 (q, J_{C,F}=320.0 Hz, C-Tf), 169.6 (C═O), 169.9 (C═O), 170.2 (C═O); ^{19F}NMR (CDCl₃, 235.3 MHz): δ (ppm): −74.5; MS (HR-ESI) calculated for C₁₅H₂₀N₃O₁₁F₃SNa [M+Na]⁺ 530.0668. Found: 530.0480.

### 2′-azidoethyl-6-O-triflate-2,3,4-tri-O-acetyl-α-D-mannopyranoside 7b

Yield: 70%; R_{f} 0.32 (Hex/AcOEt 60:40); oil; [α]_{D}=+42.9 (1, CHCl₃); IR: film (v, cm⁻¹): 2939, 2107, 1755, 1416, 1372, 1218; ^{1H}NMR (CDCl₃, 400 MHz) δ (ppm): 1.99 (s, 3H, OAc), 2.08 (s, 3H, OAc), 2.15 (s, 3H, OAc), 3.44 (ddd, 1H, J_{8, 8′}=13.4 Hz, J_{8,7}=5.8 Hz, J_{8,7′}=3.3 Hz, H8), 3.50 (ddd, 1H, J_{8′,7′}=7.0 Hz, J_{8′,7}=3.3 Hz, H8′), 3.68 (ddd, 1H, J_{7,7′}=10.5 Hz, H7), 3.87 (ddd, 1H, H7′), 4.15 (ddd, IH, J_{5,4}=10.0 Hz, J_{5,6′}=6.0 Hz, J_{5,6}=2.4 Hz, H5), 4.51 (dd, 1H, J_{6,6′}=11.3 Hz, H6), 4.58 (dd, 1H, H6′), 4.88 (d, 1H, J_{1,2}=1.5 Hz, H1), 5.22 (app t, 1H, J_{3,4}=10.0 Hz, H4), 5.26 (dd, 1H, J_{2,3}=3.4 Hz, H2), 5.38 (dd, 1H, H3); ^{13C}NMR (CDCl₃, 100 MHz) δ (ppm): 21.0 (2C, OAc), 21.2 (OAc), 50.7 (C-8), 66.2 (C-4), 67.7 (C-7), 68.8 (C-3), 68.9 (C-5), 69.5 (C-2), 74.3 (C-6), 98.0 (C-1), 119.0 (q, J_{C,F}=320.0 Hz, C-Tf), 170.0 (C═O), 170.3 (C═O), 170.4 (C═O); ^{19F}NMR (CDCl₃, 235 MHz) δ (ppm): −74.6; MS (HR-ESI) calculated for C₁₅H₂₀N₃O₁₁F₃SNa [M+Na]⁺ 530.0668. Found: 530.0659.

General Operating Mode for the Synthesis of Compounds 8 Using a Tosylate as Leaving Group

To a solution of 0.27 mmol (100 mg) of compound 6 in 2 mL of dichloromethane are added 0.1 mL of triethylamine and 2 eq (105 mg, 0.54 mmol) of tosyl chloride. The solution is left under agitation 3 hours at ambient temperature. The reaction mixture is evaporated under reduced pressure. The crude is purified on a silica column (Cyclohexane/ethyl acetate 60:40).

The following compounds are obtained:

### 2′-azidoethyl-6-O-tosyl-2,3,4-tri-O-acetyl-β-D-glucopyranoside 8a

Yield: 78%; R_{f} 0.5, Hexane/AcOEt 6:4; solid, mp=114° C., [α]_{D}=−15.4 (1, CH₂Cl₂); IR: film (n, cm⁻¹): 2943, 2106 (N3), 1756, (C═O), 1218. ^{1H}NMR (CDCl₃, 400 MHz) δ (ppm): 1.97 (s, 3H, OAc), 1.98 (s, 3H, OAc), 2.02 (s, 3H, OAc), 2.45 (S, 3H, Me-Ts), 3.24 (ddd, 1H, J_{8,7′}=3.4 Hz, J_{8,7}=4.9 Hz, J_{8,8′}=13.3 Hz, H8), 3.43 (ddd, 1H, J_{8′,7}=3.4 Hz, J_{8′,7′}=8.1 Hz, J_{8′,8}=13.3 Hz, H8′), 3.64 (ddd, 1H, J_{7′,8}=3.4 Hz, J_{7′,8′}=8.1 Hz, J_{7′,7}=10.9 Hz, H7′), 3.77 (ddd, 1H, J_{5,6}=3.4 Hz, J_{5,6′}=5.4 Hz, J_{6,4}=9.9 Hz, H5), 3.93 (ddd, 1H, J_{7,8′}=3.4 Hz, J_{7,8}=4.9 Hz, J_{7,7′}=10.9 Hz, H7), 4.03-4.11 (m, 2H, H6), 4.54 (d, 1H, J_{1,2}=7.9 Hz, H1), 4.87-4.96 (m, 2H, H2, H4), 5.17 (app t, 1H, J=9.4 Hz, H-3), 7.35 (d, 2H, J=8.3 Hz H—Ar), 7.75 (d, 2H, J=8.1 Hz, H—Ar). ^{13C}NMR (CDCl₃, 100 MHZ) δ (ppm): 20.5 (OAc), 20.5 (OAc), 20.6 (OAc), 21.6 (Me-Ts), 50.4 (C-8), 67.6 (C-6), 68.5 (C-7), 68.5 (C-4), 70.8 (C-2), 71.6 (C-5), 72.4 (C-3), 100.4 (C-1), 127.9 (2C—Ar), 129.9 (2C—Ar), 132.3 (Cq-Ar), 145.2 (Cq-Ar), 169.2 (C═O), 169.4 (C═O), 170.1 (C═O). MS ESI 529.1366, 552 (M+Na)⁺. Analysis: calculated for C₂₁ H₂₇N₃O₁₁S: C: 47.63; H: 5.13; N: 7.93; X:S: 6.05. Found: C: 48.26; H: 5.30; N: 7.80.

### 2′-azidoethyl-6-O-tosyl-2,3,4-tri-O-acetyl-α-D-mannopyranoside 8b

Yield: 68%, R_{f} 0.31, Hex/AcOEt 60:40; white solid, mp=110-111° C., [α]_{D}=+38.5; IR: film (n, cm⁻¹): 3433, 2954, 2102, 1750, 1374, ^{1H}NMR (CDCl₃, 250 MHz) δ (ppm): 1.98 (s, 3H, OAc), 1.99 (s, 3H, OAc), 2.13 (s, 3H, OAc), 2.46 (s, 3H, Me-Ts), 3.39-3.47 (m, 2H, 2H8), 3.56-3.65 (m, 1H, H7), 3.78-3.86 (m, 1H, H7′), 4.02-4.14 (m, 3H, H5, H6), 4.80 (d, 1H, J1,2=1.8 Hz, H1), 5.16 (app t, 1H, J=10.0 Hz, H4), 5.24 (dd, 1H, J_{2,3}=3.6 Hz, _{j2,1}=1.8 Hz, H2), 5.32 (dd, 1H, J_{3,4}=10.0 Hz, J_{3,2}=3.6 Hz, H3), 7.35 (d, 2H, J=8.0 Hz, H—Ar), 7.79 (d, 2H, J=8.0 Hz, H—Ar). ^{13C}NMR (CDCl₃, 62.9 MHz) δ (ppm): 20.8 (2OAc), 21.0 (OAc), 21.8 (Me-Ts), 50.5 (C-8), 66.3 (C-4), 67.3 (C-7), 68.4 (C-6), 68.8 (C-3), 68.9 (C-5), 69.4 (C-2), 97.7 (C-1), 128.3 (2C—Ar), 10.0 (2C—Ar), 132.9 (C—Ar), 145.2 (Cq-Ar), 169.9 (2C═O), 170.2 (C═O). MS ESI 529.1366, 552 (M+Na)⁺. Analysis: calculated for C₂₁H₂₇N₃O₁₁S: C: 47.63; H: 5.13; N: 7.93; X:S: 6.05. Found: C: 48.15; H: 5.26; N: 7.55; X: 5.70.

### 2′-azidoethyl-6-O-tosyl-2,3,4-tri-O-acetyl-α-D-galactopyranoside 8cα

Yield: 72%; R_{f} 0.45 (Cyclohex/AcOEt 60:40); colourless oil; IR: film (v, cm⁻¹): 2928, 2109, 1749, 1371, 1229; ^{1H}NMR (CDCl₃, 400 MHz) δ (ppm): 1.96 (s, 3H, OAc), 2.06 (s, 3H, OAc), 2.07 (s, 3H, OAc), 2.45 (s, 3H, Me-Ts), 3.34 (ddd, 1H, J_{8′8′}=13.5 Hz, J_{7′,8}=6.0 Hz, J_{7,8}=3.0 Hz, H8), 3.47 (ddd, 1H, J_{7,8′}=7.5 Hz, J_{7′,8′}=3.0 Hz, H8′), 3.56 (ddd, 1H, J_{7,7′}=11.0 Hz, H7), 3.83 (ddd, 1H, H7′), 3.05 (dd, 1H, J_{6 ,6′}=10.5 Hz,=J_{5,6}=7.0 Hz, H6), 3.30 (dd, 1H, J_{5,6′}=5.5 Hz, H6′), 4.25 (app dt, 1H, J_{5,6}=J_{5,6′}=6.0 Hz, J_{4,5}=1.0 Hz, H5), 5.08 (dd, 1H, J_{2,3}=10.5 Hz, J_{1,2}=3.5 Hz, H2), 5.12 (d, 1H, H1), 5.31 (dd, 1H, J_{3,4}=3.5 Hz, H3), 5.58 (dd, 1H, H4), 7.35 (d, 2H, J=8.0 Hz, H—Ar), 7.76 (d, 2H, J=8.0 Hz, H—Ar); ^{13C}NMR (CDCl₃, 100 MHz) δ (ppm): 20.6 (OAc), 20.7 (OAc), 20.9 (OAc), 21.8 (Me-Ts), 50.5 (C-8), 66.7 (C-5), 67.2 (C-6), 67.4 (C-3), 67.5 (C-7), 67.8 (C-2), 68.0 (C-4), 96.5 (C-1), 128.1 (2C—Ar), 130.1 (2C—Ar), 132.5 (Cq-Ar), 145.4 (Cq-Ar), 170.0 (C═O), 170.1 (C═O), 170.7 (C═O).

### 2′-azidoethyl-6-O-tosyl-2,3,4-tri-O-acetyl-β-D-galactopyranoside 8cβ

Yield: 82%; R_{f} 0.47 (Cyclohex/AcOEt 60:40); colourless oil; IR: film (v, cm⁻¹): 2933, 2099, 1744, 1-[35, 1371, 1220; ^{1H}NMR (CDCl₃, 400 MHz) δ (ppm): 1.96 (s, 3H, OAc), 2.04 (s, 3H, OAc), 2.04 (s, 3H, OAc), 2.45 (s, 3H, Me-Ts), 3.27 (ddd, 1H, J_{8,8′}=13.5 Hz, J_{7′,8}=5.0 Hz, J_{7,8}=3.5 Hz, H8), 3.48 (ddd, 1H, J_{7,8′}=8.5 Hz, J_{7′,8′}=3.5 Hz, H8′), 3.65 (ddd, 1H, J_{7,7′}=11.0 Hz, H7), 3.96 (app dt, 1H, J_{5,6}=6.0 Hz, J_{4,5}=1.0 Hz, H5), 3.97 (m, 2H, H6 and H7′), 4.12 (dd, 1H, J_{6,6′}=10.0 Hz, H6′), 4.52 (d, 1H, J_{1,2}=8.0 Hz, H1), 4.98 (dd, 1H, J_{2,3}=10.5 Hz, J_{3,4}=3.5 Hz, H3), 5.15 (dd, 1H, H2), 5.44 (dd, 1H, H4), 7.35 (d, 2H, J=8.5 Hz, H—Ar), 7.76 (d, 2H, J=8.5 Hz, H—Ar); ^{13C}NMR (CDCl₃, 100 MHz) δ (ppm): 20.6 (2OAc), 20.9 (OAc), 21.8 (Me-Ts), 50.6 (C-8), 66.6 (C-6), 67.0 (C-4), 68.5 (C-2), 68.7 (C-7), 70.8 (C-3), 70.9 (C-5), 101.2 (C-1), 128.1 (2C—Ar), 130.1 (2C—Ar), 132.4 (Cq-Ar), 145.5 (Cq-Ar), 169.6 (C═O), 170.1 (C═O), 170.1 (C═O).

General Operating Mode for the Synthesis of Compounds 9

To a solution of 650 mg (1.7 mmol) of compound 6 under nitrogen in 10 mL of diglyme are added 0.3 mL DAST (diethylaminosulfur trifluoride). The mixture is heated to 110° C. for 45 minutes then cooled to 0° C. and 5 mL methanol are then slowly added. The reaction mixture is concentrated under reduced pressure, diluted in CH₂Cl₂ (100 mL), washed with a saturated aqueous NaHCO₃ solution (5 mL) then with water (10 mL). The organic phase is dried over MgSO₄ and evaporated in vacuo. The crude is purified on a silica column (hexane/ethyl acetate: 70:30).

The following compounds are obtained:

### 2′-azidoethyl-6-deoxy-6-fluoro-2,3,4-tri-O-acetyl-β-D-glucopyranoside 9aβ

Yield: 70%; R_{f} 0.4 (Hex/AcOEt 60:40); white solid, mp=105° C.; [α]_{D}=−29.3 (1, CH₂Cl₂); IR: film (v, cm⁻¹): 3488, 2944, 2106, 1756, 1219, 1038; ^{1H}NMR (CDCl₃, 400 MHz) δ (ppm): 1.99 (s, 3H, OAc), 2.03 (s, 3H, OAc), 2.04 (s, 3H, OAc), 3.28 (ddd, 1H, J_{8,7}=3.4 Hz, J_{8,7′}=4.8 Hz, J_{8, 8′}=13.4 Hz, H8), 3.43-3.82 (m, 5H, H5, 2H6, H7, H8′), 4.05 (ddd, 1H, J_{7′,8′}=3.4 Hz, J_{7′,7}=10.6 Hz, H7′), 4.62 (d, 1H, J_{1,2}=7.9 Hz, H1), 5.00 (dd, 1H, J_{4,3}=9.4 Hz, J_{4,5}=5.2 Hz, H4), 5.02 (dd, 1H, J_{2,3}=9.4 Hz, H2), 5.24 (app t, 1H, H3); ^{13C}NMR (CDCl₃, 100 MHz) δ (ppm): 20.5 (2OAc), 20.6 OAc), 50.5 (C-8) 68.0 (d, J_{C,F}=6.9 Hz, C-4), 68.5 (C-7), 71.0 (C-2), 72.6 (C-3), 72.7 (d, J_{C,F}=19.7 Hz, C-5), 81.3 (d, J_{C,F}=175.0 Hz, C-6), 100.5 (C-1), 169.3 (C═O), 169.4 (C═O), 170.2 (C═O); ^{19F}NMR (CDCl₃, 235 MHz) δ (ppm): −231.2; MS (HR-ESI) calculated for C₁₄H₂₀N₃O₈FNa [M+Na]⁺ 400.1132. Found: 400.1128.

### 2′-azidoethyl-6-deoxy-6-fluoro-2,3,4-tri-O-acetyl-α-D-glucopyranoside 9aα

Yield: 60%; R_{f} 0.34 (Cyclohexane/AcOEt 60:40); white solid; mp=83° C.; [α]_{D}=+128.0 (0.2, CHCl₃); IR (film): v (cm⁻¹): 2947, 2109, 1753, 1369, 1223, 1037; ^{1H}NMR (CDCl₃, 400 MHz): δ (ppm): 2.00 (s, 3H, OAc), 2.04 (s, 3H, OAc), 2.06 (s, 3H, OAc), 3.44 (ddd, 1H, J_{8,8′}=13.5 Hz, J_{8,7′}=6.5 Hz, J_{8,7}=3.5 Hz, H-8), 3.48 (ddd, 1H, J_{8′,7}=7.0 Hz, J_{8′,7′}=3.5 Hz, H-8′), 3.63 (ddd, 1H, J_{7,7′}=10.5 Hz, H-7), 3.87 ddd, 1H, H-7′), 4.04 (ddt, 1H, J_{H,F}=23 Hz, J_{5,4}=10.0 Hz, J_{5,6}=3.5 Hz, H-5), 4.47 (dd, 2H, J_{H,F}=47.0 Hz, H-6), 4.86 (dd, 1H, J_{2,3}=10.0 Hz, J_{2,1}=3.5 Hz, H-2), 5.04 (app t, 1H, J_{4,3}=J_{4,5}=10.0 Hz, H-4), 5.13 (d, 1H, H-1), 5.51 (app t, 1H, H-3); ^{13C}NMR CDCl₃, 100.6 MHz): δ (ppm): 20.6 (OAc), 20.6 (OAc), 20.7 (OAc), 50.3 (C-8), 67.4 (C-7), 68.1 (d, J_{C,F}=7.0 Hz, C-4), 68.4 (d, J_{C,F}=19.5 Hz, C-5), 69.9 (C-3), 70.5 (C-2), 81.3 (d, J_{C,F}=174.5 Hz, C-6), 95.9 (C-1); ^{19F}NMR (CDCl₃, 235.3 MHz): δ (ppm): −232.20 (dt, JF,6(′)=47.0 Hz, JF,5=23.0 Hz); MS (HR-ESI) calculated for C₁₄H₂₀N₃O₈FNa [M+Na]⁺ 400.1127. Found: 400.11433.

### 2′-azidoethyl-6-deoxy-6-fluoro-2,3,4-tri-O-acetyl-α-D-mannopyranoside 9b

Yield: 80%; R_{f} 0.38 (Hex/AcOEt 60:40); oil; [α]_{D}=+42.9 (1, CHCl₃); IR: film (v, cm⁻¹): 2942, 2107, 1753, 1372, 1247, 1221; ^{1H}NMR (CDCl₃, 400 MHz): δ (ppm): 2.02 (s, 3H, OAc), 2.09 (s, 3H, OAc), 2.17 (s, 3H, OAc), 3.45 (ddd, 1H, J_{8,7′}=3.8 Hz, J_{8,7}=6.0 Hz, J_{8,8′}=13.2 Hz, H8), 3.52 (ddd, 1H, J_{8′,7}=3.8 Hz, J_{8′,7′}=7.0 Hz, H8′), 3.65-3.73 (m, 1H, H7), 3.91 (ddd, 1H, J_{7,7′}=10.5 Hz, H7′), 4.00-4.12 (m, 1H, H5), 4.42-4.51 (ddl, 2H, J_{H,F}=47.0 Hz, J_{6,5}=4.5 Hz, 2H6), 4.90 (d, 1H, J_{1,2}=1.5 Hz, H1), 5.30 (dd, 1H, J_{2,3}=3.5 Hz, H2), 5.31 (app t, 1H, J_{3,4}=J_{4,5}=10.0 Hz, H4), 5.38 (dd, 1H, H3); ^{13C}NMR (CDCl₃, 100 MHz) δ (ppm): 21.1 (OAc), 21.1 (OAc), 21.3 (OAc), 50.7 (C-8), 65.9 (d, J_{C,F}=6.3 Hz, C-4), 67.5 (C-7), 69.2 (C-3), 69.7 (C-2), 70.0 (d, J_{C,F}=19.0 Hz, C-5), 81.9 (d, J_{C,F}=176.0 Hz, C-6), 98.1 (C-1), 170.2 (C═O), 170.2 (C═O), 170.5 (C═O); ^{19F}NMR (CDCl₃, 235 MHz) δ (ppm): 231.6; MS (HR-ESI) calculated for C₁₄H₂₀N₃O₈FNa [M+Na]⁺ 400.1132. Found: 400.1131.

General Operating Mode for the Synthesis of Compounds 11

The method is identical to the one used for the preparation of compounds 4.

The following compounds are obtained:

### 2′-azidoethyl-6-deoxy-6-fluoro-β-D-glucopyranoside 11aβ

Yield: 67%; oil; [α]_{D}=−92.1 (0.76, H₂0); IR: KBr (v, cm⁻¹): 3393, 2924, 2108, 1346, 1288; ^{1H}NMR (D₂O, 400 MHz) δ (ppm): 3.15-3.25 (m, 1H, H2), 3.38-3.58 (m, 5H, 2H8, H5, H4, H3), 3.71-3.78 (m, 1H, H7), 3.90-3.97 (m, 1H, H7′), 4.44 (d, 1H, J_{1,2}=7.6 Hz, H1), 4.62 (dd, 2H, J_{H,F}=47.5 Hz, J_{6,5}=2.1 Hz, 2H6); ^{13C}NMR (D₂O, 100 MHz) δ (ppm): 50.9 (C-8), 68.6 (d, J_{C,F}=6.9 Hz, C-4), 69.0 (C-7), 73.3 (C-2), 74.8 (d, J_{C,F}=17.4 Hz, C-5), 75.8 (C-3), 82.2 (d, J_{C,F}=168.8 Hz, C-6), 102.8 (C-1); ^{19F}NMR (D4-MeOH, 235 MHZ) δ (ppm): −235.5; MS (HR-ESI) calculated for C₈H₁₄N₃O₅FNa [M+Na]⁺ 274.0815. Found: 274.0827.

### 2′-azido-6-deoxy-6-fluoro-α-D-glucopyranoside 11 aα

Yield: 95%; R_{f} 0 (Cyclohexane/AcOEt 60:40); white solid; mp=70° C.; [α]D32 +91.8 (0.2, H₂O); IR (KBr): v (cm⁻¹): 3426, 2933, 2113, 1281, 1026; ^{1H}NMR (D₂O, 400 MHz): δ (ppm): 3.48 (ddd, 1H, J_{8,8′}=13.5 Hz, J_{8,7}=6.0 Hz, J_{8,7′}=3.0 Hz, H-8), 3.53 (dd, 1H, J_{4,5}=10.0 Hz, J_{4,3}=9.5 Hz, H-4), 3.59 (dd, 1H, J_{2,3}=9.5 Hz, J_{2,1}=4.0 Hz, H-2), 3.58-3.65 (m, 1H, H-8′), 3.73 (ddd, 1H, J_{7,7′}=11.0 Hz, J_{7,8′}=3.0 Hz, H-7), 3.77 (app t, 1H, J_{3,4}=9.5 Hz, H-3), 3.89 (dddd, 1H, J_{H,F}=29.0 Hz, J_{5,6′}=3.5 Hz, J_{5,6} =2.0 Hz, H-5), 3.92 (ddd, 1H, J_{7′,8′}=7.5 Hz, H-7′), 4.70 (ddd, 1H, J_{H,F}=48.0 Hz, J_{6,6′}=11.0 Hz, H-6), 4.76 (ddd, 1H, J_{H,F}=47.0 Hz, H-6′), 5.00 (d, 1H, H-1); ^{13C}NMR (D₂O, 100.6 MHz): δ (ppm): 50.3 (C-8), 66.8 (C-7), 68.3 (d, J_{C,F}=7.0 Hz, C-4), 70.7 (d, J_{C,F}=17.5 Hz, C-5), 71.1 (C-2), 72.7 (C-3), 82.1 (d, J_{C,F}=168.0 Hz, C-6), 98.4 (C-1); ^{19F}NMR (D₂O, 235.3 MHz): δ (ppm): −235.2 (dt, J_{F,8(′)}=48.0 Hz, J_{F,5}=29.0 Hz); MS (HR-ESI) calculated for C₈H₁₄N₃O₅FNa [M+Na]⁺ 274.0810. Found: 274.0805.

### 2′-azidoethyl-6-deoxy-6-fluoro-α-D-mannopyranoside 11b

Yield: 83%; oil; [α]_{D}=+46.6 (1, Me0H; IR: film (v, cm⁻¹): 3392, 2931, 2107, 1443, 1285; ^{1H}NMR (D₂O, 400 MHz) δ (ppm): 3.49 (ddd, 1H, J_{8,7′}=3.5 Hz, J_{8,7}=6.4 Hz, J_{8,8′}=13.5 Hz, H8), 3.55 (ddd, 1H, J_{8′,7}=3.1 Hz, J_{8′,7′}=6.4 Hz, H8′), 3.72 (ddd, 1H, J_{7,7′}=10.8 Hz, H7′), 3.77-3.83 (m, 1H, H4), 3.79-3.88 (m, 1H, H5), 3.84-3.89 (m, 1H, H3), 3.91 (ddd, 1H, H7), 4.00 (dd, 1H, J_{2,3}=3.2 Hz, J_{1,2}=1.5 Hz, H2), 4.61-4.84 (m, 2H, 2H6), 4.93 (d, 1H, H1); ^{13C}NMR (D₂O, 100 MHz) δ (ppm): 50.5 (C-8), 65.7 (d, J_{C,F}=7.1 Hz, C-4), 68.9 (C-7), 70.2 (C-2), 70.6 (C-3), 71.9 (d, J_{C,F}=17.3 Hz, C-5), 82.6 (d, J_{C,F}=168.0 Hz, C-6), 100.5 (C-1); ^{19F}NMR (D₂O, 235 MHz) δ (ppm): −234.4; MS (HR-ESI) calculated for C₈H₁₄N₃O₅FNa [M+Na]⁺ 274.0815. Found: 274.0804.

II-Labelling of the Prosthetic Groups

[symbol] Manual Synthesis

[symbol]

To 7.3 mg of precursor 7 (0.023 mmol) dissolved in 400 μL of CH₃CN are added 250 μL of [¹⁸F]F⁻[P₂EtH]⁺ (11.9 mCl) and 15 μL of Barton base. The reaction mixture is heated to 120° C. for 5 to 10 min (for glucose β and mannose a respectively). The incorporation of [¹⁸F]F⁻ is verified by Radio-TLC (radiochemical purity: 66 to 71% of mannose and glucose respectively). The radiochemical yield corrected for decay is 63% for mannose a and 66% for glucose β, much higher than the yields obtained for some prior art compounds.

The preceding [¹⁸F]10 solution is diluted with 15 mL of water and the [¹⁸F]10 compound loaded on a Waters C18 cartridge. After loading, base hydrolysis using 2N NaOH is performed for 7 min at ambient temperature (25° C.). The activity is then deprotected with 2.5 ml of water then 500 μL of 2N NaOH. The efficacy of deprotection is verified by Radio-TLC (silica gel, ACN/H₂O: 90:10); radiochemical purity is higher than 97%. The deprotection yield is 75% for glucose β and the total radiochemical yield of [¹⁸F]12 corrected for decay is 50% for glucose β. The use of MeONa instead of NaOH allows improved deprotection yield.

In addition, deprotection with MeONa followed by neutralisation with ascorbic acid allows for a faster process, the generation of sodium ascorbate being useful for the subsequent click reaction.

The following compounds are obtained;
2′-azidoethyl-6-deoxy-6-[¹⁸F]fluoro β-D-glucopyranoside 12a
2′-azidoethyl-6-deoxy-6-[¹⁸F]fluoro-α-D-mannopyranoside 12b

[symbol] Automated Synthesis

[symbol]

Automated radiolabelling can also be carried out using an All-in-One automated instrument by Trasis for example.

The [¹⁸F]F⁻ is trapped on a QMA Sep Pak light cartridge which is then eluted with 1 mL of CH₃CN/H₂O solution containing K₂₂₂ and K₂CO_{3.} The solution is evaporated under a stream of nitrogen, the fluorine thus being dried. The precursor 7 is then added in dry acetonitrile and the solution heated to 95° C. for 900 s. The mixture is then passed through a silica sep pack to remove inter alia the fluorides which have not reacted. Radio-TLC performed at this step shows radiochemical purity of 100% and incorporation yields varying from 32 to 45% according to sugar. The product is then deprotected with a strong base (MeONa) and neutralised with hydrochloric acid solution. Radio-TLC shows radiochemical purity of 100% and deprotection yields of 95%. The global yield of radiosynthesis varies from 31 to 43% depending on the sugar used. In particular, for glucose β a yield of 45% is obtained for labelling and 95% for deprotection (MeONa) i.e. a global yield of 43%. For glucose α, a yield of 32% is obtained for labelling and a yield of 95% for deprotection (MeONA) i.e. a global yield of 31%. For mannose α the yield is 40% for labelling and 95% for deprotection (MeONa) i.e. a global yield of 38%.

These results show that the molecules of the present invention allow very high fluorine 18 incorporation yields to be obtained independently of sugar geometry, whilst maintaining the stereochemistry of the said sugar.

Additionally, whether the spacer arm is α or β has little influence on the yield of labelling.

III: Coupling of the Prosthetic Groups with Model Peptides

Preparation of S-propargyl-L-glutathion

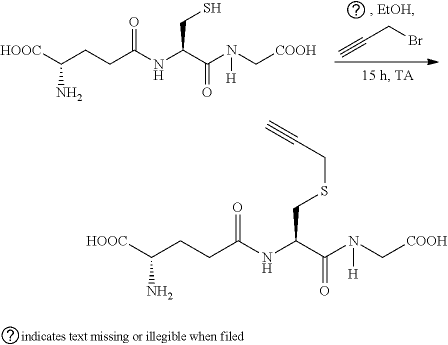

To a solution of L-glutathion 1.5 mmol (460 mg) in 10 mL of an aqueous 1:1 mixture of methanol-concentrated ammonia at 0° C. are added 1.6 mmol (195 mg) of propargyl bromide in 0.5 mL methanol. After an agitation time of 1 h at 0° C. the mixture is concentrated in vacuo at 40° C. The residue re-dissolved in 5 mL of water and lyophilised.

Yield: 81%; white solid; [α]_{D}=22.9 (1.5, H₂O); ^{IH}NMR (D₂0, 400 MHz) δ (ppm): 2.07 (dd, 2H, J=7.5 Hz, J=15.0 Hz, H-β Glu), 2.45 (m, 2H, H-γ Glu), 2.60 (dd, 1H, J=2.5 Hz, H-alkyne), 2.92 (dd, 1H, J=9.0 Hz, J=14.5 Hz, H-β 3.18 (dd, 1H, J=5.0 Hz, H-β′ Cys), 3.29 (m, 2H, H-γ Cys), 3.70 (m, 3H, H-α Glu, H-α Gly), 4.65 (dd, 1H, H-α Cys); ^{13C}NMR (D₂O) 100 MHz) δ (ppm): 18.9 (C-y Cys), 26.2 (C-β Glu), 31.4 (C-y Glu), 32.7 (C-β Cys), 43.4 (C-α Gly), 52.8 (C-α Cys), 54.1 (C-α Glu), 72.5 (CH alkyne), 80.3 (C alkyne), 171.8 (C═O), 173.9 (C═O), 174.9 (C═O), 176.2 (C═O); MS (HR-ESI) calculated for C₁₃H₁₈N₃O₆S [M−H]⁻ 344.0922. Found: 344. 0925.

Preparation of a Labelled Glycopeptide

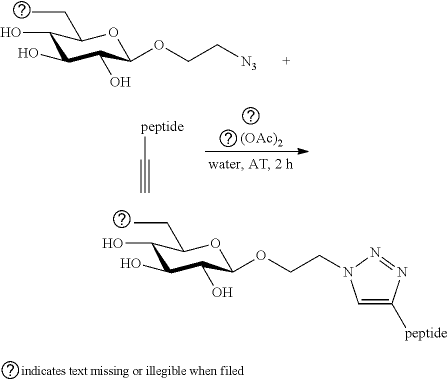

### L-glutathion-S[[1-[2-[(6-deoxy-6-[¹⁸F]fluoro-β-D-glucopyranosyl)oxy]ethyl]-1H-1,2,3-triazol-4-yl]methyl] [¹⁸F]14

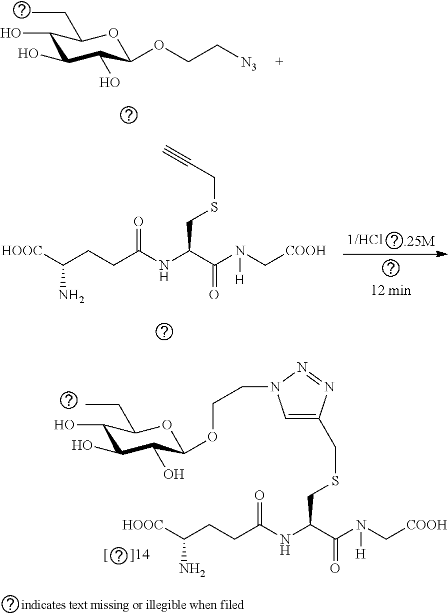

To the solution of [¹⁸F]12a prepared previously is added 230 μL of 0.25 M HCl solution to bring the pH to 8-9. The addition is then made of 2 mg of propargylated peptide, 125 μL of 0.6 M sodium ascorbate solution and 125 μL of 0.6 M Cu(OAc)₂ solution, and the whole is heated 12 min at 60° C. The efficacy of coupling is verified by Radio-TLC and the yield is 44%.

Purification by sep-pack allows the removal after the labelling step of the fluorides which have not reacted and the copper at the end of the synthesis.

The adding of a spacer arm facilitates the reaction with the propargylated peptides for example, the spacer arm allowing the sugar to be distanced from the peptides in the radiotracer obtained.

Preparation of Arg-Gly-Asp-Cys (S-propargyl) or (S-propargyl RGDC)

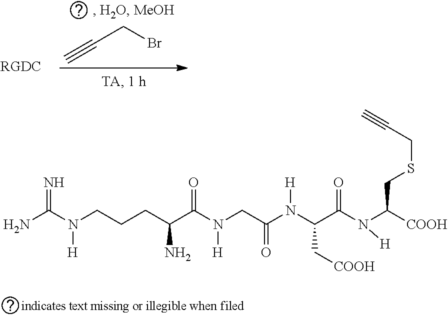

To a solution of RGDC (34 mg, 0.0075 mmol) in water (0.5 mL) 25% ammonia is first added (0.45 mL) at 0° C. followed by a solution of freshly distilled propargyl bromide (9.9 mg, 0.082 mml) in MeOH (0.25 mL). The reaction is left under agitation 3 h at ambient temperature and then concentrated under reduced pressure, followed by the addition of water and lyophilisation of the solution.

Yield: 85%; white solid; [α]_{D}=+1.0 (2, H₂O); ^{1H}NMR (D₂O, 250 MHz) δ (ppm): 1.68 (m, 2H), 1.92 (m, 2H), 2.55 (dd, 1H, J=8.0 Hz, J=16.0 Hz), 2.63 (m, 1H, CH alkyne), 2.70 (dd, 1H, J=5.0 Hz, J=16.0 Hz), 2.99 (dd, 1H, J=7.0 Hz, J=14.0 Hz), 3.10-3.35 (m, 5H), 3.75-4.25 (m, 3H), 4.35-4.40 (m, 1H), 4.40-4.50 (m, 1H); MS (ESI) 488 [M+H]⁺ 510 [M+Na]⁺ 541 [M+K]⁺.

Automated Preparation of a Labelled Glycopeptide: (S-propargyl-RGDC Coupled with the Prosthetic Group Glucose α)

Radiosynthesis is conducted using an All-In-One synthesizer (Trasis).

The [18F]F⁻ is trapped on a QMA Sep Pak light and then eluted with 1 mL of CH₃CN/H₂Osolution containing K₂₂₂ and K₂CO₃. The solution is evaporated under a stream of nitrogen, the fluorine thereby being dried. The precursor (glucose triflate α, 7aα) is added in dry acetonitrile and the solution heated to 95° C. for 900 s. The mixture is then passed through a silica sep pack to remove inter alia the fluorides which have not reacted and the product is deprotected with a strong base (MeONa). The solution is then neutralised with ascorbic acid solution thereby generating sodium ascorbate useful for the next step. The solution is transferred to a second reactor containing S-propargyl-RGDC. To this is added Cu(OAc)₂ and the mixture heated for 800 s at 65° C. The solution is transferred to a chelex cartridge to remove the copper and is then collected in a penicillin type bottle. Radio-TLC is performed and shows radiochemical purity of 100%, the coupling yield is 33%.

1-13. (canceled)

14. A compound of formula (I) 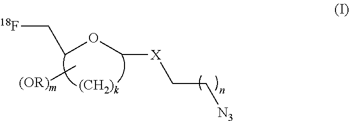
where:
k equals 2 or 3;
n is an integer between 1 and 5;
R is independently H, a C₁-C₅ alkyl group, m being an integer between 0 and 2 if k=2 and m between 0 and 3 if k=3; and
X is selected from the group comprising O, S, CH₂, NR′ where R′ is independently a C₁-C₅ alkyl group, an aryl group,
including all the stereoisomers thereof.

15. The compound of claim 1, of formula (II): 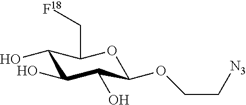
including all the stereoisomers thereof.

16. A method for synthesising the compound of claim 14, comprising the steps of:
forming, on a hexopyranose or pentofuranose compound and at anomeric position, a C₂-C₆ alkyl spacer arm terminating in an azide group;
inserting a leaving group at 6-position if k=3 or at 5-position if k=2 and protector groups on the other positions;
incorporating fluorine at 6-position if k=3 or at 5-position if k=2; and
deprotecting the other positions.

17. The method of claim 16, wherein the insertion of a leaving group at 6-position if k=3 or at 5-position if k=2 and of protector groups at the other positions comprises the inserting of a first protector group at 6-position if k=3 or at 5-position if k=2 and of a second protector group at the other positions, the deprotection of the first protector group at 6-position if k=3 or at 5-position if k=2 by a hydroxyl group and the inserting of the leaving group at 6-position if k=3 or at 5-position if k=2.

18. The method of claim 17, wherein the first protector group if a trityl ether and the second protector group is an acetate.

19. A method for synthesising the compound of claim 15, comprising the steps of:
forming, on a hexopyranose or pentofuranose compound and at anomeric position, a C₂-C₆ alkyl spacer arm terminating in an azide group;
inserting a leaving group at 6-position if k=3 or at 5-position if k=2 and protector groups on the other positions;
incorporating fluorine at 6-position if k=3 or at 5-position if k=2; and
deprotecting the other positions.

20. The method of claim 19, wherein the insertion of a leaving group at 6-position if k=3 or at 5-position if k=2 and of protector groups at the other positions comprises the inserting of a first protector group at 6-position if k=3 or at 5-position if k=2 and of a second protector group at the other positions, the deprotection of the first protector group at 6-position if k=3 or at 5-position if k=2 by a hydroxyl group and the inserting of the leaving group at 6-position if k=3 or at 5-position if k=2.

21. The method of claim 20, wherein the first protector group if a trityl ether and the second protector group is an acetate.

22. The method according to any of claims 16 to 21, wherein the leaving group is selected from the group comprising tosylate and triflate.

23. The method according to any of claims 16 to 21, wherein the hexopyranose or pentofuranose compound is selected from among hexopyranoses, pentofuranoses and the anomeric acetates thereof.

24. An intermediate molecule of formula (Ill): 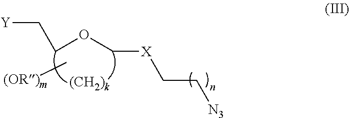
where:
Y is independently F, ¹⁸F;
R″ is selected so that OR″ forms a second protector group;
k is 2 or 3;
n is an integer between 1 and 5.
m is an integer between 0 and 2 if k=2 and between 0 and 3 if k=3;
X is selected from the group comprising O, S, CH_{2,} NR′ where R′ is independently a C₁-C₅ alkyl group, an aryl group
including all the stereoisomers thereof.

25. The intermediate molecule of claim 24 of formula (IV): 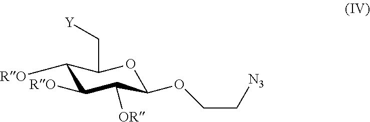
where:
R″ is an acetyl group;
Y is independently F, 18F,
including all the stereoisomers thereof.

26. An intermediate molecule of formula (V): 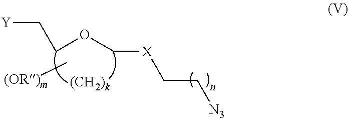
where:
is independently a tosylate leaving group, a triflate leaving group;
R″ is an acetyl group;
k is 2 or 3;
n is an integer between 1 and 5;
m is an integer between 0 and 2 if k=2 and between 0 and 3 if k=3;
X is selected from the group comprising O, S, CH_{2,} NR′ where R′ is independently a C₁-C₅ alkyl group, an aryl group;
including all the stereoisomers thereof.

27. The intermediate molecule of claim 26 of formula (VI): 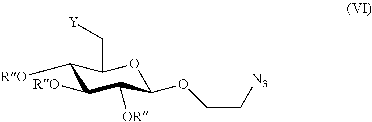
where:
R″ is an acetyl group;
Y is independently a tosylate leaving group, a triflate leaving group;
including all the stereoisomers thereof.

28. The compound according to any of claims 14 and 15 for use as prosthetic group intended to be coupled to a biomolecule by cycloaddition of its azide group with a terminal alkyne group provided on the said biomolecule.

29. The use of a compound according to any of claims 14 and 15 for the radiolabelling of a biomolecule on which there is provided a terminal alkyne group.
